(19) United States
(12) Patent Application Publication
(10) Pub. No.: US 2009/0017506 A1
(43) Pub. Date: Jan. 15, 2009

(54) CONTINUOUS FERMENTATION PROCESS TO PRODUCE BACTERIAL CELLULOSIC SHEETS

(75) Inventors: Luiz Fernando Xavier Farah, Curitiba (BR); Pablo Angel Sanchez Podlech, Joinville (BR); Cristiane do Rocio Archanjo, Curitiba (BR); Lucila Adriani Coral, Curitiba (BR)

Correspondence Address: VOLPE AND KOENIG, P.C., UNITED PLAZA, SUITE 1600, 30 SOUTH 17TH STREET, PHILADELPHIA, PA, 19103, US

(73) Assignee: BIONEXT PRODUTOS BIOTECNOLOGICOS LTDA., SAO PAULO (BR)

(21) Appl. No.: 11/722,532

(22) PCT Filed: Dec. 22, 2005
(86) PCT No.: PCT/BR05/00259

§ 371 Date: Jun. 20, 2008

(30) Foreign Application Priority Data
Dec. 22, 2004 (BR) PI 0405990-5

(51) Int. Cl. C12P 19/04

(52) U.S. Cl. 435/101

(57) ABSTRACT

A process to obtain multiple bacterial cellulose sheets without the need to replace new fermentation medium in the tray is provided. The medium is prepared and medium components in water are dissolved. The medium is sterilized and heated to 115° C. for 20 minutes, cooled to 30° C., inoculated with a suspension of bacteriae, and distributed in the trays that are left to rest. The sheets are harvested and drained over the corresponding trays. Further steps include repetition of the fermentation in the trays left to rest and harvest of the sheets from the trays, draining them over the corresponding trays until the volume of medium in the tray is 15%-25% of the initial volume. Then, a new medium should be added. Fermentation and draining is repeated until the volume of medium in the tray is 15%-25% of the initial volume, when new medium is added and the cycle is re-started.

The present invention refers to a continuous industrial production process for bacterial cellulosic sheets with high purity and specific chemical and physical properties. The bacterial celluloses sheet can be used for various purposes.

Any of the culture media mentioned in the literature (e.g. Biochemical Journal, Vol. 58, pp. 345-352, 1956; Brazilian Journal of Chemical Engineering, vol. 13, pp. 47-50, 1996; Carbohydrate Polymers, vol. 40, pp. 137-143, 1999; Journal of Bioscience and Bioengineering, vol. 88, pp. 183-188, 1999; Biotechnology and Bioengineering, vol. 68, pp. 345-372, 2000; Biotechnology and Applied Biochemistry, vol. 35, pp. 125-132, 2002) may be used for the continuous process, object of the present invention. Obviously, working conditions will depend on the fermentation media and microorganisms used. The numeric results, presented below just as examples, were obtained from a strain selected from Acetobacter xylinum and a fermentation medium containing glucose (10 to 50 g/l), yeast extract (0.5 to 4.0 g/l), monopotassium phosphate (0 to 2.0 g/l), heptahydrated magnesium sulphate (0 to 1.5 g/l) and ethyl alcohol (0.5 to 2.0% by volume). The experiments were made under the temperature of 30±2° C. on 50 cm×30 cm×12 cm trays (interface area between the fermentation medium and air, 1500 cm²).

The first references to cellulose produced by bacteriae go back to 1886 by Brown, A. J. on Journal of Chemical Society, Tarr and Hibert in 1931, Khouvine in 1936 and Hestrin, Aschener and Mager in 1947 published studies on fermentation media aiming at cellulose production. Khausal and Walker published in 1951 a study also presenting different fermentation media for cellulose production.

In 1977, Clovin published a study describing the effects of the addition of glucose to the fermentation medium for cellulose production. Clovin also mentioned the formation of a sheet composed of cellulose fibrils that become visible after 6 to 8 hours of medium inoculation. In a publication of 1980, Clovin reported the extrusion of cellulose by the bacteria membrane, stating that the microfibrils spontaneously assemble in the culture medium to form a cellulose fibril. Lepard et al, in a publication of 1975, reported that growing microfibrils are linearly extended polyglucosane structures, in principle highly hydrated and up to 100 nm wide.

Later on, still in suspension on the liquid medium, they gradually associate to form a consolidated fibril. According to the literature and observations made, such cellulose fibrils randomly associate, structuring a sheet that floats on the fermentation medium.

For decades, bacterial cellulose has been produced in Far Eastern countries (e.g. the Philippines and Thailand) for the production of “coconut cream” (a sweet obtained by cooking cellulosic sheet in sugar syrup). In the process used, the medium is water or coconut milk and the fermentation lasts weeks. After fermentation, the sheet is harvested and the fermentation medium is replaced by a new medium to begin a new cycle. Furthermore, since it is a manual process, there is no control of the fermentation temperature and environment air humidity, thus resulting in a product with variable quality. As a result, it is difficult to implement an economically viable industrial operation where the quality of the product can be assured, due to the lack of regularity in the conditions and period of fermentation of the raw material.

Patent BR PI 9204232 discloses environmental conditions to obtain cellulosic sheets, with the requirement of air renewal at a flow of 5 m³/h for a sheet surface equal to 1 m². Such technology presents the inconveniences of high operational cost (e.g. frequent change of absolute air filters) and the need of strict and complex controls for various parameters (e.g. control of air humidity) generating high production costs. Furthermore, it mentions the formation of a lamella adhered to the lower face of the sheet, with different characteristics from the sheet and which must be removed.

Patents BR 8404937 of 1984 and U.S. Pat. No. 4,912,049 of 1990 describe a fermentation medium to produce sheets by Acetobacter xylinum, where the source of nitrogen is an extract of Tea sinensis and the source of carbon is sucrose. It is currently known that Acetobacter xylinum uses glucose as a carbon source. The use of sucrose by said patent slows down the reproduction of the bacteria, increasing the time of fermentation to obtain the sheet, since the hydrolysation of sucrose into glucose and fructose is required.

Borzani and Souza (in a paper published in Biotechnology Letters, vol. 17, pp. 1271-1272, 1995) demonstrated that the cellulosic sheet is formed in its interface with air and not in its interface with the medium.

Patent application BR PI 0205499-0 describes the production of bacterial cellulose from a given fermentation medium in covered trays. In this case, the process comprises:

1. preparation of the fermentation medium;
2. inoculation of the medium;
3. filling of the fermentation trays;
4. fermentation;
5. collection of the sheets formed; and
6. change of used fermentation trays for clean ones.

This process is discontinuous and presents considerable “dead times” (items 3, 5 and 6) between one batch and another. The following case for example: a) 200 trays are being used for each batch; b) the fermentation time to obtain fine pellicles of a given gramatura (weight of a paper sheet in grams per square meter) is 48 hours; c) two people carry out all the operations for each batch. The first “dead time” of about three hours in the example presented here is spent with the distribution of trays in the chamber, that provides appropriate conditions of temperature and air circulation and their filling with inoculated fermentation medium (1.5 liters per tray). After 48 hours of fermentation, the formed sheets will be harvested and the trays with residual media will be taken from the chamber. This is the second “dead time” which, in this example, is also of about three hours. A full batch will therefore take 54 hours, from which six (12.5% of the fermentation time) are “non productive hours”. Furthermore, about 300 liters of inoculated medium should be prepared and 200 trays should be washed and prepared for another batch every 48 hours.

Bearing in mind said inconveniences and aiming to fulfill a gap existing on the market, the present invention was developed. The present invention refers to a continuous production process for sheets, minimizing dead times, reducing labor and increasing productivity of the process. By the process of the present invention, multiple bacterial cellulose sheets are obtained with no need to replace a new fermentation medium in the tray.

Experiments for the production of cellulosic sheets by bacteriae made on trays and in industrial scale have shown that, if the volume and the composition of the fermentation medium placed in the tray follow given requirements, the tray will be able to produce several sheets, all of them with practically the same content of cellulose, without replacement of the nutrients consumed by the bacteriae (see Example 1).

The present invention will be better understood by looking at the attached figures, given as mere examples, but not limiting:

FIG. 1—schematic representation of the continuous process with the production of (M) sheets per tray without re-feeding of the tray with non inoculated new medium;

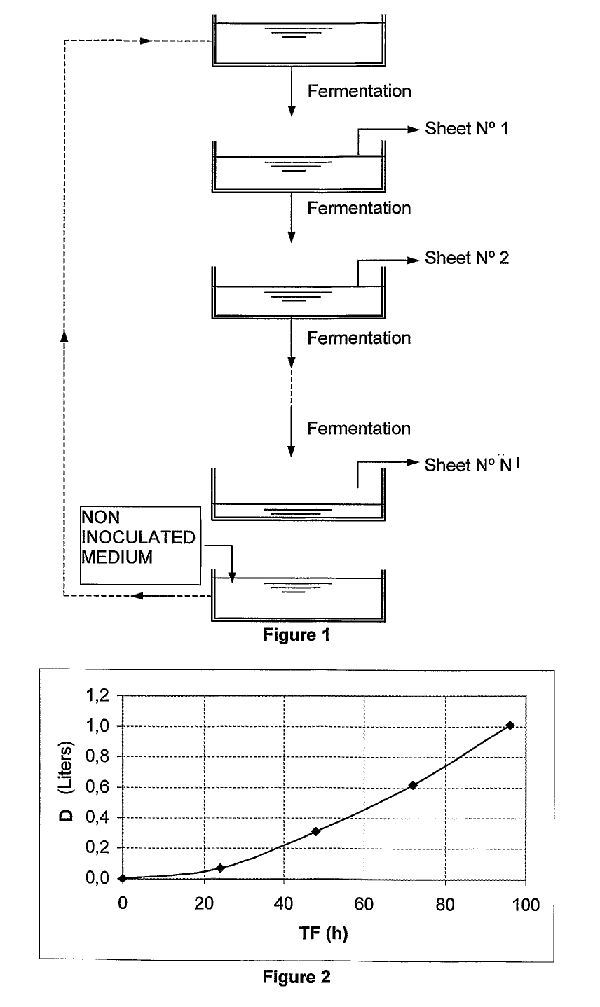

FIG. 2—medium volume reduction graph (D) in the tray when a sheet is harvested whose formation time is TF;

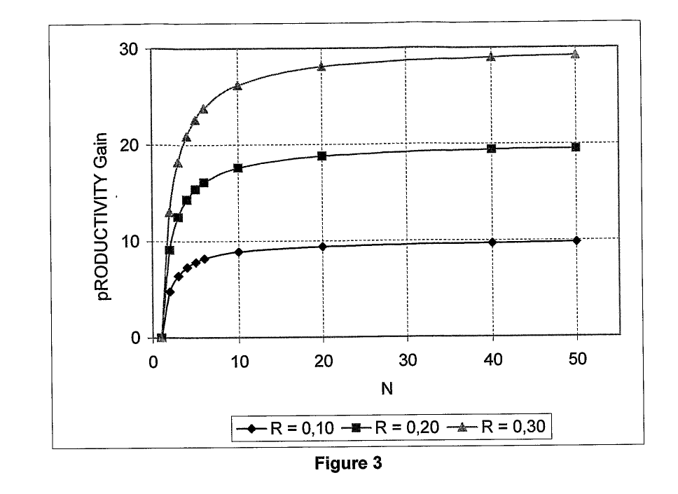

FIG. 3—productivity increase graph (P_{C}/P_{D}) regarding the ratio between “dead time”/fermentation time (R) and the total number of sheets that may be produced in a tray (N).

The continuous process for the production of cellulosic sheets, object of the present invention, comprises the following steps:

a) preparation of the fermentation medium; dissolution of medium components in water;
b) sterilization of the medium: heating of the medium to 115° C., keeping it at this temperature for 20 minutes;
c) cooling of the medium until its temperature reaches 30° C.;
d) medium inoculation with a suspension of bacteriae.
e) distribution of the medium in the trays;
f) fermentation: the trays are left to rest (the fermentation time depends on the gramatura of the final pellicle desired);
g) harvest of the sheets, draining them over the corresponding trays;
h) repetition of the fermentation in the trays left to rest during fermentation and harvest of the sheets from the trays, draining them over the corresponding trays until, after collecting and draining the sheet, the volume of medium in the tray is between 15% and 25% of the initial volume;
i) when the volume of 15% to 25% of the initial volume is reached, a new fermentation medium should be added to the tray (prepared according to the initial fermentation medium, i.e. dissolution of medium components in water; medium sterilization; heating of the medium to 115° C., keeping it at that temperature for 20 minutes; cooling the medium until its temperature reaches 30° C.) in order to obtain the initial volume again; and
j) repetition of the fermentation in the trays left to rest during fermentation and collecting the sheets from the trays, draining them over the corresponding trays until, after draining the sheet, the volume of medium in the tray is between 15% and 25% of the initial volume when new fermentation medium is added and the cycle is re-started.

As in any continuous process, the sequence of cycles described in item “j” is not indefinitively extended. Interruptions in the process may be caused by various reasons, among them we can mention: 1) equipment maintenance and repair; 2) formation of filaments in suspension on the medium, frequently formed during fermentation, and whose accumulation may even damage the formation of sheets; 3) accidental contamination of the medium by harmful microorganisms.

The main advantages of the continuous process, object of this invention, over the discontinuous process, may be observed in Example 2, which compares the results of experiment made in the conditions indicated in Example 1, and the results obtained in the production of sheets by a discontinuous process.

The additional information required to understand the following steps of the continuous process are also described here, as follows: sheet draining, preparation of inoculum, fermentation time and volume of medium in the tray and its variation.

### 1. Sheet Draining

The cellulose in the sheet taken from the trays, which will form the final pellicle, represents less than 20% of the mass of the sheet. In a sheet of approximately 600 g, for instance, there is only 3 to 4 g of cellulose. The rest of the sheet is medium impregnated. On the other hand, said water medium that impregnates the sheet is much more concentrated in bacteriae (5 to 10 times more) than the free medium in the tray. For these reasons, it is important to recover part of this medium, both to prepare the inoculum and for the continuous process, object of this invention.

Sheet draining is the operation to recover part of the water medium that permeates the sheet.

As an example, Table 1 shows the approximate volume of water medium recovered by draining the sheet.

TABLE 1

| Mass of the sheet(g) | Volume of drained medium (ml) |
|---|---|
| 100 to 200 | 200 to 300 |
| 300 to 400 | 400 to 600 |
| 600 to 700 | 400 to 600 |
| 900 to 1100 | 400 to 600 |

### 2. Preparation of the Inoculum

Two cases should be considered as follows:

1) preparation of the inoculum from a pure culture of bacteriae or lyophilized bacteriae;
2) preparation of the inoculum from the residual water medium existing in the tray after the fermentation is finished.

In any of the cases mentioned, the volumes of medium used, temperature and incubation time depend on the composition of the medium and the microorganism used. Considering that there are many cultivation media described in the literature, and numerous cellulose producing bacteriae known, it is not possible to propose one single detailed method to prepare the inoculum applicable to all cases.

The methods described in summary below, presented here just as mere examples, have achieved good results in many cases. However, we should stress that, no matter the method used, it is essential to take due care to avoid contamination of the medium by microorganisms present in the work environment.

The preparation of the inoculum (first case) from a pure culture of bacteriae or lyophilized bacteriae comprises the following steps:

a) inoculation of a container with culture medium with bacteriae from a pure culture or lyophilized ones;
b) incubation for 48 hours;
c) stirring of the sheet formed and the rest of the medium using a glass rod (said stirring substitutes in part the draining of the small sheet formed);
d) inoculation of four Erlenmeyer flasks containing 100 to 120 ml of the fermentation medium each, with 10 to 15 ml portions of the water medium (rich in bacteriae) obtained from item c,
e) incubation of the four flasks for 48 hours;
f) stirring of the contents of the four flasks with a glass rod;
g) inoculation of a tray containing 1200 to 1300 ml of the fermentation medium with the water medium of the four flasks;
h) incubation of the tray for 48 hours;
i) taking the sheet formed, draining it over the residual medium left in the tray;
j) the water medium obtained in the previous item will serve as inoculum for other trays (the volume of inoculum should be equal to 10%-20% of the volume of fermentation medium in the trays to inoculate).
k) repetition of the tray incubation cycle for 48 hours; taking the sheet formed, draining it over the residual medium present in the tray; using the water medium obtained as inoculum until a sufficient volume of water medium (10%-20% of the volume of fermentation medium in the trays to inoculate) rich in bacteriae to inoculate the volume of fermentation medium to be used for industrial production is obtained.

In the second case, as long as due care is taken for industrial production, the preparation of the inoculum for a new batch of fermentation medium involves simply the operation mentioned in item “i” (taking the sheet formed, draining it over the residual medium existing in the tray) with the appropriate number of production trays.

### 3. Fermentation Time

The fermentation time to form a tray that, when submitted to final treatments, produces a cellulose pellicle of a given gramatura, depends on the composition of the fermentation medium, the bacteria strain and the temperature.

Experiments made with the fermentation medium and the bacteria already cited, and at the temperature of 30° C.±1° C., reached the results presented in Table 2.

TABLE 2

| G (g/m²) | TF (hours) |
|---|---|
| 6 to 8 | 24 |
| 17 to 20 | 48 |
| 30 to 33 | 72 |
| 52 to 56 | 120 |
| 210 to 230 | 456 |

G = gramatura of the final cellulose pellicle
TF = fermentation time to form the sheet

Considering the average values of gramaturas for each fermentation time, i.e.: G=7.2 g/m² (for TF=24 hours), G=19.0 g/m² (for TF=48 hours), G=31.4 g/m² (for TF=72 hours), G=55.0 g/m² (for TF=120 hours), G=220 g/m² (for TF=456 hours), the approximate value of gramatura for different fermentation time values can be calculated with the following equation:

G=−4.4+0.4922.TF

### 4. Volume of Medium in the Tray and its Variation

In the continuous process, object of this invention, the volume of medium in the tray is reduced with time for two reasons: 1) loss by evaporation; 2) loss by the harvest of the sheet formed, even draining it.

Loss by evaporation mainly depends on the temperature, the humidity of the air that circulates in the chamber storing the trays, speed of said air over the trays and the interface area between the sheet being formed and the air. The speed of the evaporation loss is usually expressed in milliliters of medium per day and per square meter of the area of the sheet/air interface. Increase in temperature, reduction of air humidity and increase in air speed in the chamber cause increase in the speed of evaporation loss, thus the control of said parameters to reach a practically constant and relatively low value of said speed is of utmost importance.

Speeds of evaporation loss of 250 ml/(m²·day) and 2,200 ml/(m²·day) were found in industrial units, which shows the impossibility of adopting an average value for the calculation.

The reduction of the volume of the medium in the tray as a consequence of the harvest of a sheet formed there will be proportional to the mass of the sheet. The bigger the sheet, the bigger will be the reduction. On the other hand, the mass of the formed sheet will be proportional to the area of the sheet/air interface and the required time of fermentation to form a sheet.

Experiments made with those already mentioned fermentation medium and bacteria in trays have shown that the reduction of the volume of medium in the tray (D) as a consequence of the harvest of a sheet corresponding to time for fermentation TF varies as shown in Table 3 and FIG. 2.

TABLE 3

| TF (hours) | D (liters) |
|---|---|
| 24 | 0.070 |
| 48 | 0.310 |
| 72 | 0.620 |
| 96 | 1.010 |

The equation that correlates D and TF, represented by the curve of FIG. 2 is:

D=0.000160.(TF)^{1.93}

If the interface area were A (in cm²), the equation would be:

$$D = \frac{A}{1500} \cdot 0.000160 \cdot {( TF )}^{1.93}$$

Example 3 shows how to calculate the volume of inoculated medium to be put in a tray, for the continuous process, object of this invention, to enable harvest of a given number of sheets corresponding to a given fermentation time, with no need to replace, in the tray, new fermentation medium (see FIG. 1).

### EXAMPLE 1

From a tray containing 10 liters of fermentation medium, it was possible to obtain in 28 days 14 sheets (48 hours of fermentation per sheet) which, after being submitted to final treatments, supplied dry cellulose pellicles with gramatura of 18 g/m², and the consumption of the medium components by the producing bacteriae did not damage the characteristics of the final product.

### EXAMPLE 2

Two hundred trays are used to produce sheets, whose fermentation time is 48 hours, by the continuous process (see Example 1) and by the discontinuous process. The results are as follows:

### 1. Continuous Process

a) each tray produces 14 sheets in 28 days, with no need to replace the medium in the trays reaching a total of 2,800 sheets produced;
b) the initial volume of inoculated medium in each tray is 10 liters;
c) the total initial volume of inoculated medium to be prepared is 2,000 liters;
d) there is only one “dead time” of three hours to prepare the trays;
e) the total fermentation time to produce 14 sheets per tray is 672 hours (14×48 hours);
f) there is only one “dead time” of three hours to unload and take off the trays;
g) the total production time of 2800 sheets is 678 hours (3+672+3);
h) process productivity is 2800/678=4.13 sheets per hour (or 99.1 sheets per day).

### 2. Discontinuous Process

a) each tray produces one sheet in 48 hours, which is then unloaded and substituted by another tray;
b) the initial volume of inoculated medium in each tray is 1.5 liters;
c) the volume of inoculated medium to be prepared for the production of 200 sheets (one per tray) is 300 liters and, for the production of 2800 sheets, is 4200 liters. In this case, there are 14 operations to prepare 300 liters of inoculated medium;
d) there are 14 “dead times” of three hours each to prepare the trays, thus resulting in a total of 42 hours (14×3);
e) the total fermentation time is also 672 hours (14×48);
f) there are 14 “dead times” of three hours each to unload and take off the trays, thus resulting in a total of 42 hours (14×3);
g) the total production time of 2800 sheets is 756 hours (42+672+42);
h) process productivity is 2,800/756=3.70 sheets per hour (or 88.9 sheets per day).

Table 4 summarizes the values presented in Example 2. We should highlight two important points in Example 2: the number of sheets produced per tray in the continuous process, without unloading trays and substituting them for others, is surely higher than 14, which increases the advantages of the continuous process over the discontinuous one; 2) The volume and polluting load of effluents in the continuous fermentation process are lower than in the discontinuous process.

TABLE 4

| | Discontinuous | Continuous |
|---|---|---|
| | process | process |
| Total volume of inoculated medium | 4.200 | 2.000 |
| (liters) | | |
| Number of operations to prepare the | 14 | 1 |
| inoculated medium | | |
| Number of loading operations of trays | 14 | 1 |
| Number of unloading operations of trays | 14 | 1 |
| Total “dead” time to load trays (hours) | 42 | 3 |
| Total “dead” time to unload trays (hours) | 42 | 3 |
| Total time of fermentation (hours) | 672 | 672 |
| Total time of production (hours) | 756 | 678 |
| Number of draining operations = number | 2.800 | 2.800 |
| of produced sheets | | |
| Productivity (sheets/hour) | 3.70 | 4.13 |
| Productivity (sheets/day) | 88.9 | 99.1 |

### EXAMPLE 3

From one tray, we intend to harvest ten drained sheets corresponding to a fermentation time of 72 hours, without replacing consumed nutrients. The speed of losses by evaporation is 500 ml/(m²·day). After harvesting the tenth sheet, the residual volume of the medium in the tray should be equal to 15% of the initial volume. Calculate the volume of inoculated medium to be put in the tray.

a) volume of medium taken from the tray with the ten sheets:

10×0.62=6.2 liters

b) volume of medium lost by evaporation:

0.15(m²)×500 mL(m²·day)×30(days)=2250 mL=2.25 liters

c) being V the initial volume of inoculated medium, the residual volume after harvest the ten sheets should be of 0.15 V;
d) the value of V will then be:

V=6.2+2.25+0.15V

V=9.94 liters≈10 liters

The continuous process, object of this invention, presents, in summary, the following advantages over the discontinuous process:

reduction of labor;

increase in productivity;

reduction of the volume of fermentation effluents;

reduction of the polluting load of fermentation effluents.

Important remark: the numerical values presented are valid for the fermentation medium and bacteria strain used in the experiments. However, this does not invalidate the above mentioned conclusions.

We should present the deduction of the equation to calculate how many times the productivity of the continuous process, object of this invention, is higher than the discontinuous process.
(TF)=Fermentation time to form the sheet
(TM)=“dead time” spent in tray loading and unloading operation
R=(TM)/(TF)=Ratio between “dead time” and fermentation time
N=number of sheets that may be produced in a single tray, by the continuous process without the need to unload the tray and substitute it for another one

In the continuous production process of N sheets, there will be only one loading operation, one unloading operation and N successive fermentations. If we indicate by T_{C} the total time to produce N sheets, we have:

T_{C}=(TM)+N*(TF)

But as (TM)=(R*(TF)), the result is:

T_{C}=(TF)×(R+N)

Thus, the productivity of the continuous process (P_{C}) will be:

$${P}_{C} = \frac{N}{{T}_{C}} = \frac{N}{( TF ) \times ( R + N )}$$

In the production of N sheets by the discontinuous process, there will be N loading operations, N unloading operations and N consecutive fermentations. In this case, the total time to produce N sheets (indicated by T_{D}) will be:

T_{D}=Nx(TF)+Nx(TF)

And remembering that: (TM)=R (TF), the result is that:

T_{D}=Nx(TF)(1+R)

The productivity of the discontinuous process (P_{D}) will then be:

$${P}_{D} = \frac{N}{{T}_{D}} = \frac{1}{( TF ) \times ( 1 + R )}$$

By dividing the value of P_{C} by the value of P_{D}, we have:

$$\frac{{P}_{C}}{Pd} = ( 1 + R ) \cdot \frac{N}{N + R}$$

Knowing the values of N and R, the last equation allows us to calculate how many times the productivity of the continuous process is higher than the discontinuous one. Table 5 and FIG. 3 show the results obtained for different values of N and R.

TABLE 5

| | Increase in Productivity (%) | | |
|---|---|---|---|
| N | R = 0.10 | R = 0.20 | R = 0.30 |
| 1 | 0.0 | 0.0 | 0.0 |
| 2 | 4.8 | 9.1 | 13.0 |
| 3 | 6.4 | 12.5 | 18.2 |
| 4 | 7.3 | 14.3 | 20.9 |
| 5 | 7.8 | 15.4 | 22.6 |
| 6 | 8.2 | 16.1 | 23.8 |
| 10 | 8.9 | 17.6 | 26.2 |
| 20 | 9.4 | 18.8 | 28.1 |
| 40 | 9.7 | 19.4 | 29.0 |
| 50 | 9.8 | 19.5 | 29.2 |

The last equation shows that, no matter how high the value of N is, P_{C}/P_{D} will never be higher than (1+R). Therefore, if R=0.20 for example, i.e. the “dead time” of loading and unloading the tray is 20% of the fermentation time, the increase in productivity when substituting the discontinuous for the continuous process will be of 20% (see Table 5 and FIG. 3).

1. Continuous fermentation process to produce bacterial cellulosic sheets, by wherein the obtainment of multiple bacterial cellulosic sheets without the need to replace new fermentation medium in the tray, said process comprising the following steps:
a) preparation of the fermentation medium; dissolution of medium components in water;
b) sterilization of the medium: heating of medium to 115 C, keeping it at that temperature for 20 minutes;
c) cooling of the medium until its temperature reaches 30 C;
d) medium inoculation with a suspension of bacteriae;
e) distribution of the medium in the trays;
f) the trays are left to rest during fermentation;
g) harvest of the sheets, draining them over the corresponding trays;
h) repetition of the fermentation in the trays left to rest during fermentation and harvest of the sheets from the trays, draining them over the corresponding trays until, after taking and draining the sheet, the volume of medium in the tray is between 15% and 25% of the initial volume;
i) when the volume of 15% to 25% of the initial volume is reached, a new fermentation medium should be added to the tray in order to reach the initial volume again; and
j) repetition of the fermentation in the trays left to rest during fermentation and harvest of the sheets from the trays, draining them over the corresponding trays until, after taking and draining the sheet, the volume of medium in the tray is between 15% and 25% of the initial volume when new fermentation medium is added and the cycle is re-started.

2. Process, according to claim 1, wherein the inoculum is prepared from a pure culture of the bacteria or lyophilized bacteria.

3. Process, according to claim 1, wherein the inoculum is prepared from the residual water medium existing in the tray in fermentation.

4. Process, according to claim 1, wherein the volumes of medium used, the temperature and the time of incubation depend on the composition of the medium and the microorganism used.

5. Process, according to claim 1, wherein the volume of inoculated medium to be put in each tray varies between 25 and 100% of the volume of the tray.

6. Process, according to claim 1, wherein the time of fermentation depends on the composition of the fermentation medium, strain and temperature.

7. Process, according to claim 1, wherein the fermentation time varies from 24 to 456 hours for a cellulose pellicle gramatura that varies from 6 to 230 g/m².

8. Process, according to claim 1, wherein the mass of the sheet taken from the trays varies from 100 to 1100 g.

9. Process, according to claim 1, wherein the cellulose existing in the sheet taken from the trays represents less than 20% of the mass of the sheet.

10. Process, according to claim 1, further comprising the step of recovering part of the water medium that impregnates the sheet.

11. Process, according to claim 10, further comprising the step of recovering from 200 to 600 ml of the volume of water medium for a mass of sheet that varies from 100 to 1100 g.

12. Process, according to claim 1, wherein the reduction of the volume of medium in the trays is as high as the mass of the sheet taken off.

13. Process, according to claim 10, wherein the water medium serves as inoculum for other trays, being its volume equal to 10 to 20% of the volume of the fermentation medium.

14. Process, according to claim 1, wherein the mass of the sheet formed is bigger when the sheet/air interface area and the fermentation time required to form the sheet are increased.

15. Process, according to claim 1, further comprising the step of reducing the volume of effluents and the load of their pollutants.

16. Process, according to claim 1, further comprising the step of reducing the cost by reducing operations and raw material.

17. Process, according to claim 1, further comprising the step of improving productivity of the process in at least 15% over the discontinuous process.
